(19) United States
(12) Patent Application Publication
(10) Pub. No.: US 2023/0339790 A1
(43) Pub. Date: Oct. 26, 2023

(54) OZONE WATER DECOMPOSITION APPARATUS AND METHOD

(71) Applicant: SEMES CO., LTD., Chungcheongnam-do (KR)

(72) Inventors: Myung A JEON, Jeollanam-do (KR); Young Seop CHOI, Chungcheongnam-do (KR); Bok Kyu LEE, Chungcheongnam-do (KR); Kyu Hwan CHANG, Chungcheongnam-do (KR); Yong Sun KO, Gyeonggi-do (KR)

(21) Appl. No.: 17/985,038

(22) Filed: Nov. 10, 2022

(30) Foreign Application Priority Data
Apr. 20, 2022 (KR) 10-2022-0048873

(51) Int. Cl. C02F 1/78

(52) U.S. Cl. CPC C02F 1/78; C02F 2301/08; C02F 2305/023; C02F 2103/346

(57) ABSTRACT

Provided are an ozone water decomposition apparatus and method that can decompose ozone water quickly and stably. The ozone water decomposition apparatus, comprising: a tank configured to accommodate ozone water and decompose the ozone water; a first supply unit configured to supply the ozone water to the tank; a second supply unit configured to supply an additive to the tank wherein the additive is supplied by a preset first or second supply amount; a circulation line configured to circulate the ozone water in the tank; and a concentration meter installed in the circulation line, wherein the ozone water decomposition apparatus comprises: after circulating the ozone water using the circulation line, measuring an ozone concentration of the ozone water using the concentration meter; subsequently supplying the additive of the first supply amount or the second supply amount based on the measured ozone concentration of the ozone water; and subsequently circulating the ozone water until the ozone concentration of the ozone water reaches a preset reference concentration.

### CROSS-REFERENCE TO RELATED APPLICATION

This application claims priority from Korean Patent Application No. 10-2022-0048873 filed on Apr. 20, 2022 in the Korean Intellectual Property Office, and all the benefits accruing therefrom under 35 U.S.C. 119, the contents of which in its entirety are herein incorporated by reference.

### BACKGROUND

### 1. Technical Field

The present disclosure relates to an ozone water decomposition apparatus and method.

### 2. Description of the Related Art

With the high integration of semiconductor devices, the miniaturization of circuit patterns has been rapidly advanced, and contaminants remaining on the substrate (e.g., particles, organic contaminants and metal contaminants) have a great influence on device characteristics and production yield. Accordingly, a cleaning process of removing the contaminants remaining on the substrate is performed before and after each unit process of manufacturing a semiconductor.

### SUMMARY

Meanwhile, ozone water may be used in the cleaning process. Since ozone water generated by an ozone water generator is always bypassed to a decomposition apparatus, the decomposition apparatus decomposes the ozone water at all times. When ozone concentration of ozone water in the decomposition apparatus is below or equal to a predetermined reference value, the decomposition apparatus drains ozone water. However, it takes a long time to decompose ozone water in order for the ozone concentration to be below a preset reference value, and when using a high concentration of the ozone water, its decomposition becomes more time-consuming.

Aspects of the present disclosure provide an ozone water decomposition apparatus that can decompose ozone water quickly and stably.

Aspects of the present disclosure also provide an ozone water decomposition method that can decompose ozone water quickly and stably.

The technical aspects of the present disclosure are not restricted to those set forth herein, and other unmentioned technical aspects will be clearly understood by one of ordinary skill in the art to which the present disclosure pertains by referencing the detailed description of the present disclosure given below.

### Technical Solution

According to an aspect of the present disclosure, there is provided an ozone water decomposition apparatus, comprising: a tank configured to accommodate ozone water and decompose the ozone water; a first supply unit configured to supply the ozone water to the tank; a second supply unit configured to supply an additive to the tank, wherein the additive is supplied by a preset first or second supply amount; a circulation line configured to circulate the ozone water in the tank; and a concentration meter installed in the circulation line. The ozone water decomposition apparatus comprises: after circulating the ozone water using the circulation line, measuring an ozone concentration of the ozone water using the concentration meter; subsequently supplying the additive of the first supply amount or the second supply amount based on the measured ozone concentration of the ozone water; and subsequently circulating the ozone water until the ozone concentration of the ozone water reaches a preset reference concentration.

According to another aspect of the present disclosure, there is provided an ozone water decomposition apparatus, comprising: a tank configured to accommodate ozone water and decompose the ozone water; a first supply unit configured to supply the ozone water to the tank; a second supply unit configured to supply hydrogen peroxide to the tank, wherein hydrogen peroxide is supplied by a preset first or second supply amount; a circulation line configured to circulate the ozone water in the tank; and a concentration meter installed in the circulation line and configured to measure an ozone concentration in the ozone water using UV. The ozone water decomposition apparatus comprises: sensing that the capacity of the ozone water filled in the tank corresponds to a maximum capacity; after circulating the ozone water using the circulation line, subsequently measuring the ozone concentration of the ozone water using the concentration meter; subsequently supplying an additive of the first supply amount or the second supply amount based on the measured ozone concentration of the ozone water; subsequently circulating the ozone water until the ozone concentration of the ozone water reaches a preset ozone concentration; and draining the ozone water from the tank.

According to an aspect of the present disclosure, there is also an ozone water decomposition method, comprising: supplying ozone water to a tank; subsequently circulating the ozone water using a circulation line connected to the tank; subsequently measuring an ozone concentration of the ozone water using a concentration meter installed in the circulation line; subsequently supplying an additive of a preset first or second supply amount based on the measured ozone concentration of the ozone water; circulating the ozone water until the ozone concentration of the ozone water reaches a reference concentration; and subsequently draining the ozone water that has reached the reference concentration.

Specific details of other embodiments are included in the detailed description and the drawings.

### BRIEF DESCRIPTION OF THE DRAWINGS

The above and other aspects and features of the present disclosure will become more apparent by describing in detail exemplary embodiments thereof with reference to the attached drawings, in which:

FIG. 1 is a block diagram illustrating an ozone water decomposition apparatus according to one embodiment of the present disclosure;

FIG. 2 is a block diagram illustrating the structure of a second supply unit illustrated in FIG. 1;

FIGS. 3 and 4 are diagrams describing an operation of the second supply unit illustrated in FIG. 2;

FIG. 5 is a flowchart illustrating an ozone water decomposition method according to one embodiment of the present disclosure;

FIG. 6 is a flowchart illustrating an example of step S30 of FIG. 5;

FIG. 7 is a flowchart illustrating another example of step S36 of FIG. 6;

FIG. 8 is a block diagram illustrating the ozone water decomposition apparatus according to another embodiment of the present disclosure;

FIG. 9 is a block diagram illustrating the ozone water decomposition apparatus according to another embodiment of the present disclosure; and

FIG. 10 is a diagram illustrating the effect of the ozone water decomposition apparatus according to one embodiment of the present disclosure.

### DETAILED DESCRIPTION OF THE EMBODIMENT

Hereinafter, embodiments of the present disclosure will be described with reference to the attached drawings. The merits and characteristics of the present disclosure and a method for achieving the merits and characteristics will become more apparent from the embodiments described in detail in conjunction with the accompanying drawings. However, the present disclosure is not limited to the disclosed embodiments, but may be implemented in various different ways. The embodiments are provided to only complete the disclosure of the present disclosure and to allow those skilled in the art to understand the category of the present disclosure. The present disclosure is defined by the category of the claims. Like numbers refer to like elements throughout the description of the figures.

The spatially relative terms “below”, “beneath”, “lower”, “above” and “upper” may be used to easily describe the correlation of a device or components with other devices or components. Spatially relative terms are to be understood as including terms in different directions of the device in use or operation in addition to the directions shown in the figures. For example, when flipping a device shown in the figure, a device described as “below” or “beneath” of another device may be placed “above” of another device. Thus, the exemplary term “below” can encompass both an orientation of above and below. The device can also be oriented in other directions, so that spatially relative terms can be interpreted according to orientation.

Although the first, second, etc. are used to describe various elements, components and/or sections, these elements, components and/or sections are of course not limited by these terms. These terms are only used to distinguish one element, component or section from another element, component or section. Therefore, the first device, the first component, or the first section mentioned below may be a second device, a second component, or a second section within the technical spirit of the present disclosure.

Hereinafter, embodiments of the present disclosure will be described with reference to the attached drawings. The same or similar elements are assigned the same reference numerals irrespective of their reference numerals, and a redundant description thereof is omitted.

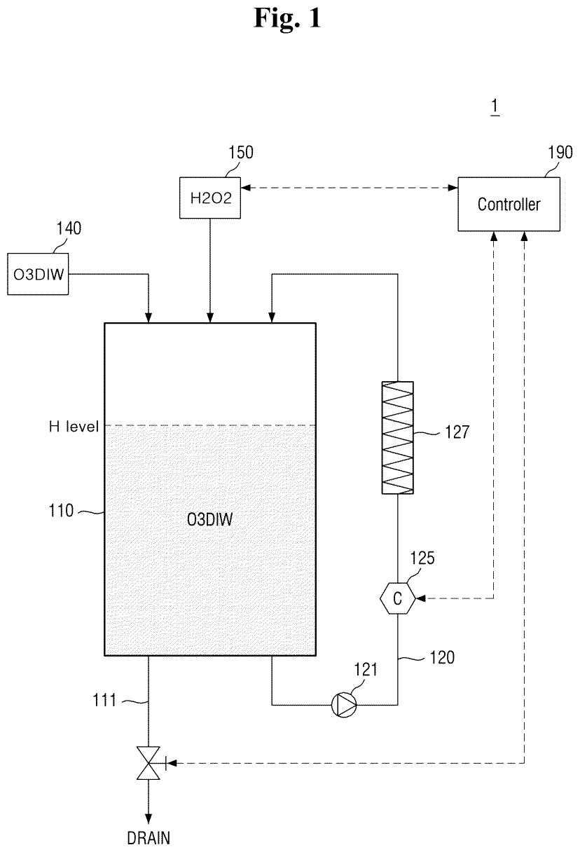

FIG. 1 is a block diagram illustrating an ozone water decomposition apparatus according to one embodiment of the present disclosure.

Referring to FIG. 1, an ozone water decomposition apparatus 1 according to one embodiment of the present disclosure includes a tank 110, a first supply unit 140, a second supply unit 150, a circulation line 120, a concentration meter 125, a circulation pump 121, a heater 127, a controller 190, and a drain line 111.

The tank 110 is a container to accommodate an ozone water (O3DIW) and decomposing the ozone water. The tank 110 may accommodate the ozone water by a preset maximum capacity (indicated by an H level in FIG. 1). When the capacity of the ozone water accommodated in the tank 110 reaches the maximum capacity, the ozone water is not supplied into the tank 110. Although this is not separately displayed, a sensor capable of checking the capacity of the accommodated ozone water is installed in the tank 110.

The first supply unit 140 supplies the ozone water to the tank 110. The first supply unit 140 may be any device capable of supplying the ozone water to the tank 110. The first supply unit 140 may be a substrate processing device (e.g., a cleaning device and a photoresist removal device), and in that case, the ozone water may be used in the substrate processing device and then drained into the tank 110. Alternatively, the first supply unit 140 may be an ozone water generator (not shown), and in that case, the ozone water generated by the ozone water generator may be supplied to the tank 110 without passing through the substrate processing apparatus (i.e., bypassing the ozone water generator). The first supply unit 140 may supply the ozone water into the tank 110 in a spray manner. For instance, the first supply unit 140 may be supplied in the spray manner from an upper side of the tank 110 to a lower side. As the ozone water flows into the tank 110 in the spray manner, it is primarily decomposed.

The second supply unit 150 supplies an additive to the tank 110. The additive is a material to assist the decomposition of the ozone water, and may include, for example, at least one of H₂O₂, IPA (isopropyl alcohol), and NH₄O₄. In addition to the aforementioned examples, any material—as long as it can assist the composition of the ozone water—can be used. Hereinafter, for convenience of description, only a case where the additive is H₂O₂ will be described.

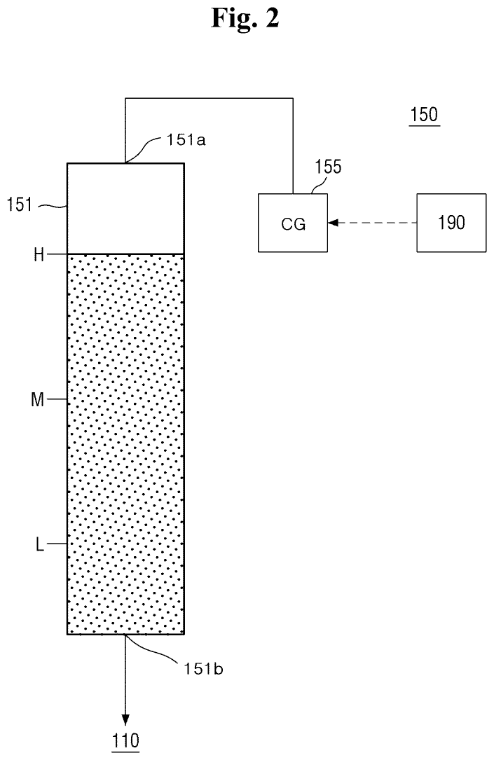

The second supply unit 150 supplies the additive, but supplies any one of preset different supply amounts. Furthermore, the supply amounts of the additive may be two different supply amounts (i.e., a first or second supply amount), or three different supply amounts (i.e., a first, second, or third supply amount). Depending on the design, the supply amounts may be four or more preset supply amounts. The configuration and operation of the second supply unit 150 will be described below with reference to FIGS. 2 to 4.

The circulation line 120 is installed in the tank 110 to circulate the ozone water (or a mixture of the ozone water and the additive).

In addition, the circulation pump 121, the concentration meter 125, and the heater 127 are installed in the circulation line 120. The ozone water is circulated along the circulation line 120 by the circulation pump 121. The concentration meter 125 measures the concentration (i.e., ozone concentration) of the ozone water. The concentration meter 125 may be a concentration meter using UV, and, for instance, the ozone concentration is calculated by reading a value at a certain wavelength. The heater 127 heats the ozone water in the circulation line 120. The ozone water is heated and circulated in the circulation line 120 and is secondly decomposed.

The controller 190 receives the ozone concentration measured by the concentration meter 125 and determines the amount of the additive to be supplied into the tank 110 according to the ozone concentration.

For instance, when the measured ozone concentration belongs to a first range, the controller 190 controls the second supply unit 150 so that the additive of the first supply amount is supplied to the tank 110.

In addition, when the measured ozone concentration belongs to a second range different from the first range, the controller 190 controls the second supply unit 150 so that the additive of the second supply amount different from the first supply amount is supplied to the tank 110.

For example, the second range may be greater than the first range, and the second supply amount may also be greater than the first supply amount.

Meanwhile, when supplying the additive (i.e., hydrogen peroxide) to the tank 110, the amount of hydrogen peroxide needs to be adjusted so that it is not excessively added. A reaction ratio of ozone (O₃) included in ozone water and hydrogen peroxide (H₂O₂) added thereto may be below 1:1.

Herein, the reaction ratio refers to a ratio in which elements or molecules of a product are required for a chemical reaction to take place. For instance, when there is 2A+B→C, a substance A has to be twice as large as a substance B so that the substances A and B react to become a substance C. In the example, the reaction ratio is 2:1. When reactants are different in volume and concentration, in the aforementioned reaction formula, the reaction ratio is “concentration of A×volume of A”:“concentration of B×volume of B.”

In order for ozone to completely react with hydrogen peroxide, the reaction ratio of ozone and hydrogen peroxide is 1:1, but a (small) amount of hydrogen peroxide may be added so that the reaction ratio does not become 1:1.

The reason for doing so is that after ozone reacts with and hydrogen peroxide, the remaining hydrogen peroxide affects the operation of the concentration meter 125 using UV. For instance, when using UV at 250 nm, absorption may occur in ozone and hydrogen peroxide. In the concentration meter 125, the concentration of an imaginary quantity may be measured by hydrogen peroxide.

As described above, when the reaction ratio of ozone (O₃) included in ozone water and hydrogen peroxide (H₂O₂) added is below 1:1, hydrogen peroxide may not remain after the reaction between ozone and hydrogen peroxide. Accordingly, it is possible to prevent erroneous measurement by hydrogen peroxide in the concentration meter 125.

The reaction ratio may be, for instance, equal to or above 1:0.8, and equal to or below 1:0.9. For example, it may be 1:0.8, 1:0.82, 1:0.85, 1:0.88, and 1:0.9.

When the reaction ratio is below 1:0.8, since the amount of hydrogen peroxide is relatively small, ozone may not be efficiently decomposed. By controlling the reaction ratio to be below 1:0.9, even if a slightly larger amount of hydrogen peroxide is added due to a process error (or a control error), it is possible to prevent the reaction ratio from exceeding 1:1.

In addition, when 40 ppm of the ozone water are used in the substrate processing device, the concentration of the ozone water flowing into the tank 110 may be below 40 ppm (e.g., 10 ppm). Therefore, the amount of hydrogen peroxide added to the tank 110 is also considerably small.

Accordingly, as the additive (i.e., hydrogen peroxide) is supplied in a small amount, it has to be precisely controlled in consideration of the reaction ratio with ozone.

In the ozone water decomposition apparatus 1 according to one embodiment of the present disclosure, the second supply unit 150 may supply the additive only in the preset supply amount using a metering tube. The additive cannot be supplied in a supply amount not set in advance. Hereinafter, the structure and the operation of the second supply unit will be described with reference to FIGS. 2 to 4.

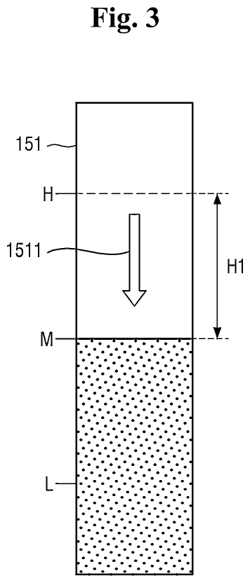

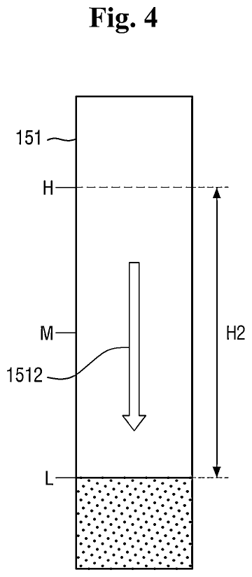

FIG. 2 is a block diagram illustrating the structure of the second supply unit illustrated in FIG. 1. FIGS. 3 and 4 are diagrams describing an operation of the second supply unit illustrated in FIG. 2.

Referring to FIG. 2, the second supply unit 150 includes a tube 151 and a control gas supply unit 155. The control gas supply unit 155 receives a control signal from the controller 190 and supplies a control gas CG to the tube 151 accordingly. As the control gas CG is a gas that does not react with the additive, it may be, for example, N₂, Ne and Ar, but the present disclosure is not limited thereto.

The additive (i.e., hydrogen peroxide) is accommodated in the tube 151. The tube 151 may be formed to extend in one direction. An inlet 151a through which the control gas is introduced is disposed on one side of the tube 151, and an outlet 151b through which the additive is discharged is disposed on the other side of the tube 151. A fluororesin group (e.g., PFA) with corrosion resistance may be used in the tube 151.

Referring to FIG. 3, when the control gas CG of a first volume is supplied into the tube 151 (see reference numeral 1511), the additive in the tube 151 is reduced from the H level (a first height) to an M level (a second height) (see reference numeral H1). The additive is supplied to the tank 110 by the first supply amount corresponding to the reference numeral H1.

Referring to FIG. 4, when the control gas CG of a second volume greater than the first volume is supplied into the tube 151 (see reference numeral 1512), the additive in the tube 151 is reduced from the H level (the first height) to an L level (a third height lower than the second height) (see reference numeral H2). The additive is supplied to the tank 110 by the second supply amount corresponding to the reference numeral H2.

In summary, in order to quantitatively supply a small amount of additives, the metering tube 151 is used to supply the additive. The supply amount (i.e., the first supply amount or the second supply amount) of the additive supplied by the metering tube 151 is set in advance. Under the control of the controller 190, when the control gas CG of the first volume is supplied, the additive of the first supply amount is supplied to the tank 110, and when the control gas CG of the second volume is supplied, the additive of the second supply amount is supplied to the tank 110.

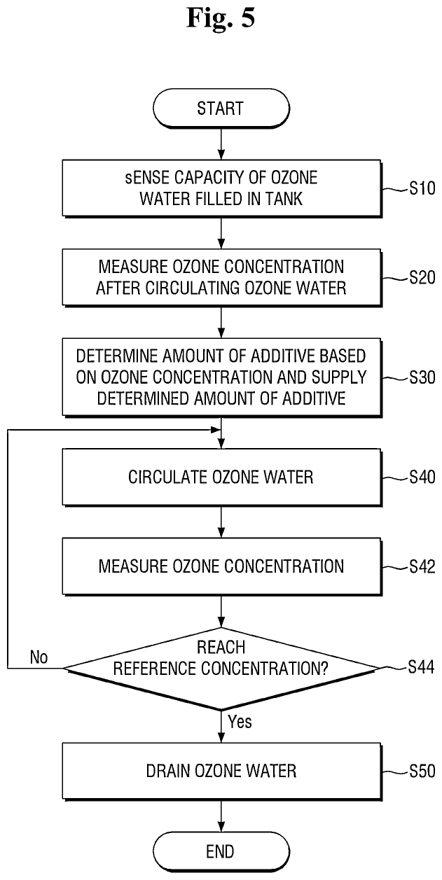

FIG. 5 is a flowchart illustrating an ozone water decomposition method according to one embodiment of the present disclosure.

Referring to FIGS. 1 and 5, first, the capacity of the ozone water filled in the tank 110 is sensed (S10).

Specifically, after the ozone water is supplied to the tank 110, the sensor (not shown) installed in the tank 110 senses whether the capacity of the ozone water filled in the tank 110 corresponds to a maximum capacity (see the H level in FIG. 1). When the capacity of the ozone water filled in the tank 110 corresponds to the maximum capacity, the ozone water is no longer supplied to the tank 110.

When the ozone water is circulated while continuously supplying the ozone water, it is difficult for the concentration meter 125 to accurately measure the ozone concentration of the ozone water. Therefore, a next step proceeds in a state where the ozone water is not supplied.

Then, after circulating the ozone water through the circulation line 120, the ozone concentration is measured using the concentration meter 125 (S20).

Specifically, before measuring the ozone concentration, a time to circulate the ozone water may be set in advance. For instance, it may be from 3 seconds to 10 seconds, but the present disclosure is not limited thereto. The concentration meter 125 measures the ozone concentration in the ozone water using UV.

Then, the amount of the additive to be added is determined based on the measured ozone concentration, and the determined amount of the additive is supplied to the tank 110 (S30).

Specifically, when the measured ozone concentration belongs to the first range, the amount of the additive to be added becomes the first supply amount. In addition, when the measured ozone concentration belongs to the second range different from the first range, the amount of the additive to be added becomes the second supply amount different from the first supply amount.

As described above, the determined amount of the additive (the first or second supply amount) is predetermined so that the additive does not remain in the ozone water after the ozone included in the ozone water reacts with the additive (hydrogen peroxide). The amount of the additive may be determined as the largest supply amount within a supply amount range in which the additive does not remain, but the present disclosure is not limited thereto.

Then, the ozone water is circulated (S40).

Specifically, the heater 127 heats the ozone water while circulating the ozone water through the circulation line 120. The ozone water is circulated and simultaneously decomposed. Under the influence of additives, the ozone water is decomposed faster.

Next, the ozone concentration is measured (S42).

Then, it is confirmed whether the measured ozone concentration has reached a reference concentration (S44).

When it does not reach the reference concentration (i.e., when the measured ozone concentration exceeds the reference concentration) (No in S44), it is circulated again (back to the step S40).

When it reaches the reference concentration (i.e., when the measured ozone concentration is below or equal to the reference concentration) (Yes in the step S44), the process proceeds to step S50.

Subsequently, the circulated ozone water is drained through the drain line 111 (S50).

Specifically, when the ozone water reaches the reference concentration (e.g., 1 ppm or 2 ppm), the ozone water is discharged through the drain line 111 without further circulating. The reference concentration may be determined by the customer's request or may be defined by relevant laws, but the present disclosure is not limited thereto.

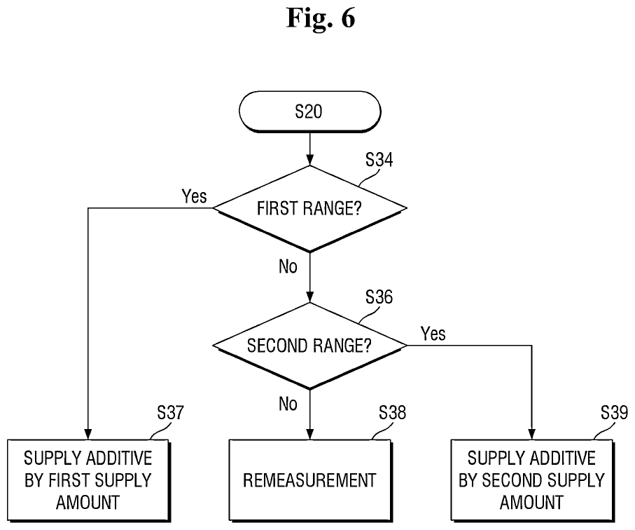

FIG. 6 is a flowchart illustrating an example of step S30 of FIG. 5.

Referring to FIG. 6, the ozone concentration of the ozone water is measured after circulating the ozone water (S20).

Then, it is confirmed whether the measured ozone concentration belongs to the first range (S34).

When the ozone concentration belongs to the first range (Yes in S34), the additive is supplied by the first supply amount (S37).

When the ozone concentration does not belong to the first range (No in S34), it is confirmed whether the measured ozone concentration belongs the second range (S36).

When the ozone concentration belongs to the second range (Yes in S36), the additive is supplied by the second supply amount (S39).

When the ozone concentration does not belong to the second range (No in S36), the ozone concentration is remeasured (S38).

For instance, the second range may exceed the first range. The first range may range from 2 ppm to 6 ppm, while the second range may range from 6 ppm to 10 ppm. In that case, the second supply amount is greater than the first supply amount.

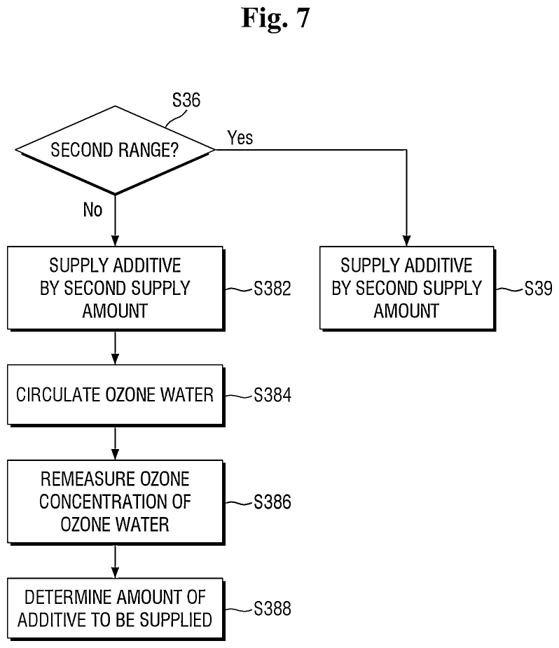

FIG. 7 is a flowchart illustrating another example of the step S36 of FIG. 6.

In FIG. 6, when the ozone concentration does not belong to the second range (Yes in S36), the additive is supplied by the second supply amount (S39), and when the ozone concentration does not belong to the second range (No in S36), the ozone concentration is remeasured (S38).

On the other hand, in FIG. 7, when the ozone concentration does not belong to the second range (for example, when it belongs to a third range greater than the second range) (No in S36), the additive is supplied by the second supply amount (S382), and the ozone water is additionally circulated by a predetermined time (S384). Since the additive is supplied, the ozone decomposition is advanced with the circulation of the ozone water.

After circulation during a predetermined period of time, the ozone concentration of the ozone water is remeasured (S386).

Then, the amount of the additive to be supplied is determined according to the remeasurement results (S388). As described above, when the ozone concentration belongs to the first range, the additive may be determined to be supplied by the first supply amount, and when the ozone concentration belongs to the second range, the additive may be determined to be supplied by the second supply amount. Alternatively, when the ozone concentration does not belong to the second range (i.e., when it belongs to the third range greater than the second range), the steps S382 to S388 are performed again.

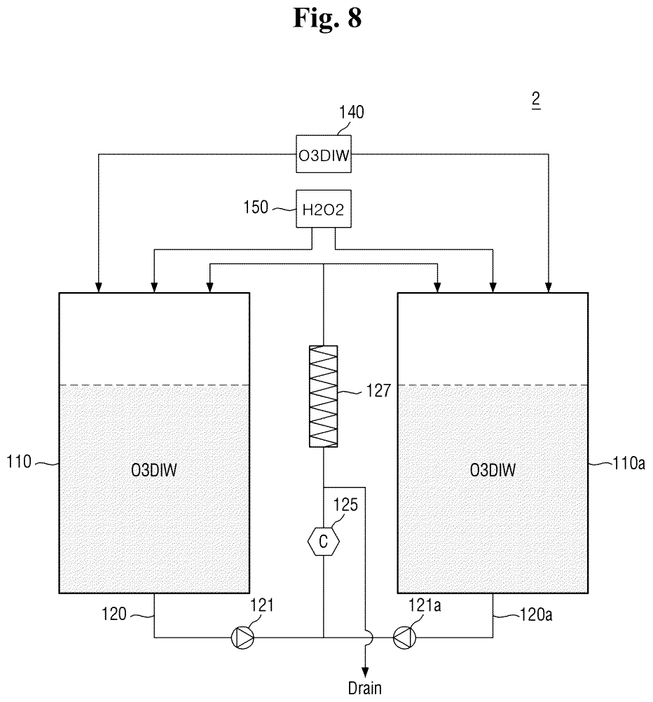

FIG. 8 is a block diagram illustrating the ozone water decomposition apparatus according to another embodiment of the present disclosure. For convenience of description, the differences from the content described with reference to FIGS. 1 to 7 will be mainly described.

Referring to FIG. 8, an ozone water decomposition apparatus 2 according to another embodiment of the present disclosure includes a plurality of tanks 110 and 110a, the first supply unit 140, the second supply unit 150, circulation lines 120 and 120a, the concentration meter 125, circulation pumps 121 and 121a, and the heater 127.

The first supply unit 140 may selectively or alternately supply the ozone water to a first tank 110 or a second tank 110a. The second supply unit 150 may selectively or alternately supply the additive (e.g., H₂O₂) to the first tank 110 or the second tank 110a. A first circulation line 120 is connected to the first tank 110, and a first circulation pump 121 is installed in the first circulation line 120. A second circulation line 120a is connected to the second tank 110a, and a second circulation pump 121a is installed in the second circulation line 120a. As illustrated, the first tank 110 and the second tank 110a may share the concentration meter 125 and the heater 127.

To explain an exemplary operation, the first supply unit 140 first supplies the ozone water to the first tank 110. When the ozone water is filled up to the maximum capacity of the first tank 110, the decomposition operation of the ozone water is performed in the first tank 110. As described above, the decomposition operation of the ozone water is meant to circulate the ozone water of the first tank 110 using the first circulation line 120 and measure its concentration using the concentration meter 125. The additive having the supply amount (e.g., the first or a second supply amount) corresponding to the concentration range of the ozone water is supplied to the first tank 110. Then, the ozone water is additionally circulated using the first circulation line 120 until the concentration of the ozone water becomes the reference concentration.

While the decomposition operation is performed in the first tank 110 in this way, the first supply unit 140 supplies the ozone water to the second tank 110a. While the ozone water is filled by the second tank 110a, the decomposition operation of the ozone water in the first tank 110 may be terminated. The ozone water decomposed in the first tank 110 is drained.

In addition, when the ozone water supplied by the first supply unit 140 is filled up to the maximum capacity of the second tank 110a, the decomposition operation of the ozone water is performed in the second tank 110a. As described above, the decomposition operation of the ozone water is meant to circulate the ozone water in the second tank 110a using the second circulation line 120a and measure the concentration of the ozone water using the concentration meter 125. The additive having the supply amount (e.g., the first or a second supply amount) corresponding to the concentration range of the ozone water is supplied to the second tank 110a. Then, the ozone water is additionally circulated using the second circulation line 120a until the concentration of the ozone water becomes the reference concentration.

As described above, the first supply unit 140 supplies the ozone water to the first tank 110 while performing the decomposition operation in the second tank 110a.

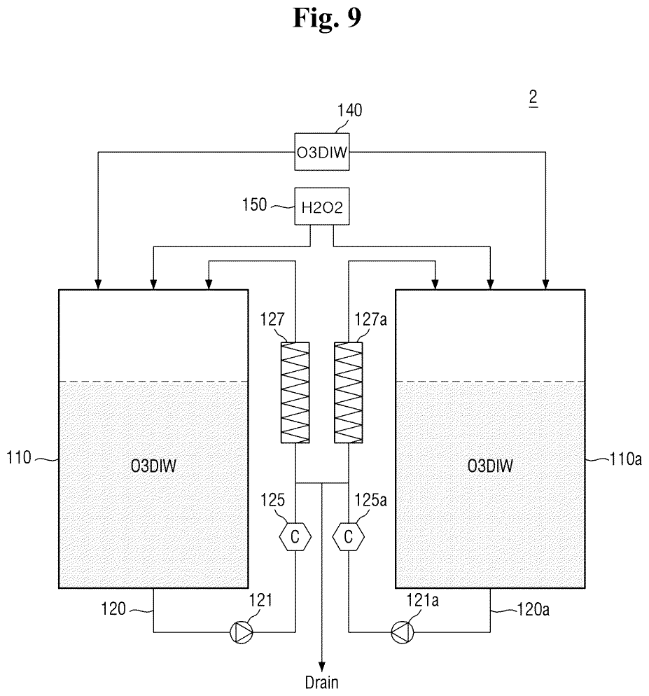

FIG. 9 is a block diagram illustrating the ozone water decomposition apparatus according to another embodiment of the present disclosure. For convenience of description, the differences from the content described with reference to FIGS. 1 to 8 will be mainly described.

Referring to FIG. 9, a first heater 127 and a first concentration meter 125 may be installed in a first circulation line 120 to correspond to the first tank 110, and a second heater 127a and a second concentration meter 125a may be installed in the second circulation line 120a to correspond to the second tank 110a.

In this way, when the circulation lines 120 and 120a, the heaters 127 and 127a, and the concentration meters 125 and 125a are separately installed in the first tank 110 and the second tank 110a, the ozone water decomposition apparatus 2 can process a larger amount of ozone water. This is because the ozone water decomposition operation may be performed in the second tank 110 even while the ozone water decomposition operation is performed in the first tank 110.

In FIG. 9, the drain line is commonly connected to the first circulation line 120 and the second circulation line 120a, but the present disclosure is not limited thereto. In other words, the drain line may also be installed for each of the circulation lines 120 and 120a.

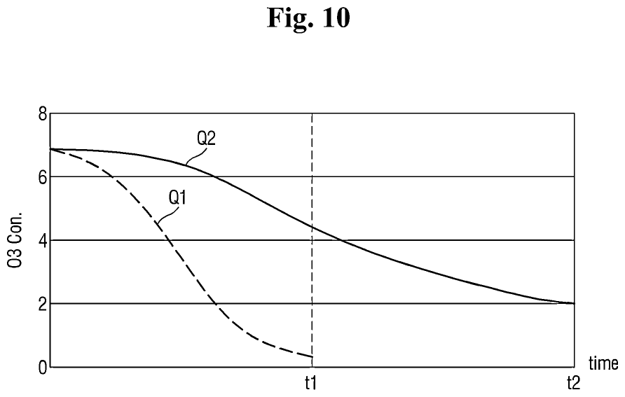

FIG. 10 is a diagram illustrating the effect of the ozone water decomposition apparatus according to one embodiment of the present disclosure.

Referring to FIG. 10, an x-axis represents time, and a y-axis represents ozone concentration.

For instance, after circulating the ozone water filled up to the maximum capacity of the tank, the ozone concentration measured using the concentration meter 125 may be 7 ppm.

Herein, when the ozone water is decomposed while circulating the ozone water using the circulation line without adding a separate additive, the ozone concentration may be 2 ppm only after t2 hours (see reference numeral Q2).

On the other hand, like the ozone water decomposition method according to one embodiment of the present disclosure, when the additive (hydrogen peroxide) corresponding to the ozone concentration is added and the ozone water is decomposed while circulating the ozone water using the circulation line, the ozone concentration can be 1 ppm or less only in t1 hours which is less than t2 (see reference numeral Q1).

Although this is not separately described, a similar effect may be created by adding IPA or NH₄OH instead of hydrogen peroxide.

Although the embodiments of the present disclosure have been described above with reference to the accompanying drawings, the present disclosure is not limited to the disclosed embodiments, but may be implemented in various different ways, and the present disclosure may be embodied in many different forms without changing technical subject matters and essential features as will be understood by those skilled in the art. Therefore, embodiments set forth herein are exemplary only and not to be construed as a limitation.

Although the embodiments of the present disclosure have been described above with reference to the accompanying drawings, the present disclosure is not limited to the disclosed embodiments, but may be implemented in various different ways, and the present disclosure may be embodied in many different forms without changing technical subject matters and essential features as will be understood by those skilled in the art. Therefore, embodiments set forth herein are exemplary only and not to be construed as a limitation.

What is claimed is:

1. An ozone water decomposition apparatus, comprising:
a tank configured to accommodate ozone water and decompose the ozone water;
a first supply unit configured to supply the ozone water to the tank;
a second supply unit configured to supply an additive to the tank, wherein the additive is supplied by a preset first or second supply amount;
a circulation line configured to circulate the ozone water in the tank; and
a concentration meter installed in the circulation line,
wherein the ozone water decomposition apparatus comprises:
after circulating the ozone water using the circulation line, measuring an ozone concentration of the ozone water using the concentration meter;
subsequently supplying the additive of the first supply amount or the second supply amount based on the measured ozone concentration of the ozone water; and
subsequently circulating the ozone water until the ozone concentration of the ozone water reaches a preset reference concentration.

2. The ozone water decomposition apparatus of claim 1, wherein the supplying the additive of the first supply amount or the second supply amount based on the measured ozone concentration of the ozone water comprises:
when the measured ozone concentration of the ozone water belongs to a first range, supplying the additive by the first supply amount; and
when the measured ozone concentration of ozone water belongs to a second range greater than the first range, supplying the additive by the second supply amount greater than the first supply amount.

3. The ozone water decomposition apparatus of claim 2, further comprising: when the measured ozone concentration of the ozone water belongs to a third range greater than the second range,
supplying the additive by the second supply amount;
circulating the ozone water by a predetermined time;
remeasuring the ozone concentration of the ozone water; and
determining the amount of the additive to be supplied based on the remeasured ozone concentration of the ozone water.

4. The ozone water decomposition apparatus of claim 1, comprising: before circulating the ozone water using the circulation line, sensing the capacity of the ozone water filled in the tank.

5. The ozone water decomposition apparatus of claim 4, wherein, when the sensed capacity of the ozone water corresponds to a maximum capacity of the tank, the ozone water is circulated using the circulation line.

6. The ozone water decomposition apparatus of claim 1, further comprising: draining the ozone water from the tank after circulating the ozone water until the ozone concentration of the ozone water reaches a preset reference concentration.

7. The ozone water decomposition apparatus of claim 1, wherein the first supply unit supplies the ozone water in a spray manner from an upper side of the tank to a lower side.

8. The ozone water decomposition apparatus of claim 1, wherein the additive includes at least one of H₂O₂, IPA, and NH₄OH.

9. The ozone water decomposition apparatus of claim 1, wherein the concentration meter is a concentration meter using UV.

10. The ozone water decomposition apparatus of claim 1, wherein the second supply unit accommodates the additive and includes a tube extending in one direction, wherein a control gas is supplied into the tube on one side of the tube, and the amount of the additive discharged to the other side of the tube is changed depending on the volume of the supplied control gas.

11. The ozone water decomposition apparatus of claim 10, wherein, when the control gas of a first volume is supplied into the tube, the additive in the tube is reduced from a first height of the tube to a second height, and
when the control gas of a second volume greater than the first volume is supplied into the tube, the additive in the tube is reduced from the first height of the tube to a third height lower than the second height.

12. An ozone water decomposition apparatus, comprising:
a tank configured to accommodate ozone water and decompose the ozone water;
a first supply unit configured to supply the ozone water to the tank;
a second supply unit configured to supply hydrogen peroxide to the tank, wherein hydrogen peroxide is supplied by a preset first or second supply amount;
a circulation line configured to circulate the ozone water in the tank; and
a concentration meter installed in the circulation line and configured to measure an ozone concentration in the ozone water using UV,
wherein the ozone water decomposition apparatus comprises:
sensing that the capacity of the ozone water filled in the tank corresponds to a maximum capacity;
after circulating the ozone water using the circulation line, subsequently measuring the ozone concentration of the ozone water using the concentration meter;
subsequently supplying an additive of the first supply amount or the second supply amount based on the measured ozone concentration of the ozone water;
subsequently circulating the ozone water until the ozone concentration of the ozone water reaches a preset ozone concentration; and
draining the ozone water from the tank.

13. The ozone water decomposition apparatus of claim 12, wherein a reaction ratio of ozone included in the ozone water and hydrogen peroxide added thereto is below 1:1.

14. The ozone water decomposition apparatus of claim 13, wherein the reaction ratio of ozone included in the ozone water and hydrogen peroxide added thereto is equal to or above 1:0.8, and equal to or below 1:0.9.

15. The ozone water decomposition apparatus of claim 12, wherein the supplying an additive of the first supply amount or the second supply amount based on the measured ozone concentration of the ozone water comprises:
when the measured ozone concentration of the ozone water belongs to a first range, supplying the additive by the first supply amount; and
when the measured ozone concentration of ozone water belongs to a second range greater than the first range, supplying the additive by the second supply amount greater than the first supply amount.

16. The ozone water decomposition apparatus of claim 15, further comprising: when the measured ozone concentration of the ozone water belongs to a third range greater than the second range,
supplying the additive by the second supply amount;
circulating the ozone water by a predetermined time;
remeasuring the ozone concentration of the ozone water; and
determining the amount of the additive to be supplied based on the remeasured ozone concentration of the ozone water.

17. An ozone water decomposition method, the method comprising:
supplying ozone water to a tank;
subsequently circulating the ozone water using a circulation line connected to the tank;
subsequently measuring an ozone concentration of the ozone water using a concentration meter installed in the circulation line;
subsequently supplying an additive of a preset first or second supply amount based on the measured ozone concentration of the ozone water;
subsequently circulating the ozone water until the ozone concentration of the ozone water reaches a reference concentration; and
subsequently draining the ozone water that has reached the reference concentration.

18. The ozone water decomposition method of claim 17, further comprising: after supplying the ozone water to the tank, sensing that the capacity of the ozone water filled in the tank corresponds to a maximum capacity.

19. The ozone water decomposition method of claim 17, wherein the measuring an ozone concentration comprises using UV.

20. The ozone water decomposition method of claim 17, wherein the supplying ozone water to a tank comprises supplying the ozone water in the spray manner from an upper side of the tank to a lower side.
